(19) United States
(12) Patent Application Publication
(10) Pub. No.: US 2011/0313041 A1
(43) Pub. Date: Dec. 22, 2011

(54) FOOD SUPPLEMENTS ON THE BASIS OF PANTOTHENIC ACID

(76) Inventor: Hans-Dieter Minge, Berlin (DE)

(21) Appl. No.: 13/141,445

(22) PCT Filed: Dec. 22, 2009
(86) PCT No.: PCT/DE2009/001793

§ 371 Date: Sep. 8, 2011

(30) Foreign Application Priority Data
Dec. 23, 2008 (DE) 10 2008 064 588.5

(51) Int. Cl. A61K 31/197; A61P 3/00; A61P 11/00; A61P 43/00; A61P 25/06; A61P 3/10; A61P 25/28; A61P 15/00; A61P 25/00; A61P 29/00; A61P 13/00; A61P 31/00; A61P 31/18; A61P 35/00; A61P 25/18; A61P 1/00; A61P 37/06; A61P 9/00; A61P 37/08; A61P 19/02; A61P 11/06; A61P 15/08; A61P 25/24; A23L 1/30; C07C 231/00

(52) U.S. Cl. 514/563; 562/569; 426/648; 426/443; 426/2

(57) ABSTRACT

The present invention relates to a dietary supplement for the treatment of weakness in metabolic activity containing Pantothenic acid (CoA), its precursors and/or its salts, a process for its production as well as its use. Application areas of the invention are food industry and medicine.

The invention is based of the surprising finding that the intake of pantothenic acid, pantothenic acid-CoA and/or its derivatives in a large amount has a beneficial effect on symptoms caused by weakness in metabolic activity.

The core point of the invention is that the uptake of a pantothenic acid-CoA equivalent from food is not 10 mg per day, but more than 10³ times of this amount to provide the human organism optimally. It was also a surprising finding that the cholinergic Panthenol-CoA uptake reaction from food was taking place within a few minutes.

The dietary supplement according to the present invention is a compound for comprising 85-99% Pantothenic acid and/or its salts and other supplements if applicable.

The present invention relates to a dietary supplement for the treatment of weakness in metabolic activity containing Pantothenic acid (CoA), its precursors and/or its salts, a process for its production as well as its use. Application areas of the invention are food industry and medicine.

### STATE OF THE ART

Weakness in metabolic activity often appears, but often underestimated, prior to diseases. It results in the development of varied symptoms of weakness, fatigue, exhaustion, listlessness, paresthesia, sensitive and mental problems in the broadest sense. Weakness in metabolic activity can lead to the emergence of diseases. All digestive organs are involved in energy generation from food.

If problems appear at this point, fatigue is often the result. In addition to organic causes, undersupply or overloading of the body may be causes of a weakness in metabolic activity and other related illnesses as well.

Thus, a decreased food intake in the reposed status of seniority associated with the corresponding habit to prefer food which is lightly denatured of substance of content of grain and rich in starch necessarily induces deficits, without to account for an increase in demand due to exposure.

In antiquity, the term melancholia characterizes the status of great sadness, melancholy, apathy. According to current understanding this refers to the so-called CFS chronic fatigue syndrome with paralyzing profound weakness, lethargy, weakness of memory, muscle weakness and gastric problems.

In contrast to the ordinary course of events from fatigue to recreation, from inanition to convalescence, this exhaustion is not reversible.

Chronic fatigue, CFS, shows weakness in metabolic activity in a permanent condition. The so-called burnout syndrome is a precursor to this and often develops to CFS.

In the event of the so-called Chronic Fatigue Syndrome (CFS) problems with the gastrointestinal tract of the digestive system can be observed, but these problems were never seen as a possible cause for CFS. (9)

The so-called Chronic Fatigue Syndrome (CFS) is a physiological state, in which the patient feels totally exhausted, although no obvious organic cause can be found. Both a very bad stamina and continuous tiredness after physical stress are two of the most typical, and weakening symptoms. In some cases the fatigue is mainly mental; in other cases it is physical. A very popular hypothesis (A) states, that metabolism of patients with CFS is normal, but fatigue and other symptoms are psychological aspects. It is said, that physical exhaustion is an absence of energy, while the mental exhaustion is a subjective sensation, which is characterized by a lack of motivation and a concern to rest. (11)

Abnormal fatigue is known as a pathological feature of many diseases, but CFS has no obvious organic cause. CFS patients are suspected of drowning in self pity of weakness, since their organs are well according to diagnostic findings. The patients find themselves in a situation, which combined with their depression leads them into psychiatric treatment, without being able to expect help.

The Chronic Fatigue Syndrome (CFS) is a multi-system disease, which deprives the victims of their dignity and health. (11)

The disease can last years. While some patients get better afterwards, others remain ill for a decade or longer without any substantial recovery. (12)

Neither the cause of weakness in metabolic activity (as especially the so-called Chronic Fatigue Syndrome (CFS)) nor a satisfying solution for its treatment has been found so far.

The extent of fatigue can be verified biochemical as a specific change of the mitochondria's function and state; a dysfunction of mitochondria is located (11) One assumes that a diminished energy generation in mitochondria causes both weakness and exhaustion (12); CFS can be initiated by viral infection of the stomach. (6) “An alternative hypothesis (B) is that there is a metabolic dysfunction with a resulting diminished energy generation. That way the dysfunction of the mitochondria leads to fatigue and can cause other symptoms of CFS.” (11)

In 2008, continuing problems in the gastrointestinal tract are still documented for veterans of the gulf war of 1991, suffering extreme exhaustion. But these problems were not considered as reason for the extreme exhaustion. (9)

Thereby the step of energy production in mitochondria has a crucial meaning. A connection between disturbances in the mitochondria and the occurrence of diseases already has been described in the state of the art (1). Especially in the development of CFS those disorders play a role. In this connection, a certain diet containing various vitamins and minerals is recommended (2). This also includes pantothenic acid. An application of pantothenic acid, is already recommended in U.S. Pat. No. 5,360,821, to improve constitution and physical strength. Also in U.S. Pat. No. 5,304,574, the addition of pantothenic acid instead of steroids in order to achieve this effect is described.

In Keil (Diss.) a connection between neurodegenerative diseases, like Alzheimer or Parkinson, and dysfunctions in the area of mitochondria is described. Thereby, a protective effect of certain substances to the mitochondria, as well as gingko biloba extract, is illuminated. (3)

### Task of the Invention

Therefore, the task of the invention is to provide a compound, which is effective against weakness in metabolic activity and eliminates its symptoms.

The task is determined by the claims 1, 2, 10-13 and 15, the sub claims are preferential variants.

### Nature of the Invention

The invention is based of the surprising finding of Mr. Hans Dieter Minge that the intake of pantothenic acid, pantothenic acid-CoA and/or its derivatives in a large amount has a beneficial effect on symptoms caused by weakness in metabolic activity.

Pantothenic acid is a vital substance occurring in plant and animal tissues from 50 to 95% in the form of coenzyme A and 4′-Phosphopantethein.

Pantothenic acid is called vitamin B5, it is soluble in water and one intakes it with food. Sources are, for example, vegetables, grains, offal, meat and fish. Pantothenic acid, in form of coenzyme A (CoA), is involved into various reactions in carbohydrate, fat and amino acid metabolism. CoA is localized at the ATP synthase, in the mitochondria, to 95%. The activated acetic acid, the acetyl-CoA, is the most important ester of coenzyme A for the intermediary metabolism. Pantothenic acid-CoA plays a central role in the modification of cellular proteins.

The usage of pantothenic acid in the patent history is geared to the recombination of CoA in the citric acid cycle. This artifice potentiates CoA to multi-varied existence in acetyl-CoA, in carbohydrate-, fat-, amino acid-, intermediary metabolism and in the synthesis of acetylcholine during minimal pantothenic acid input. The uptake of pantothenic acid-CoA into the metabolism should occur hours after the meal, after gastric passaging, simultaneously with food absorption in the small intestine. The daily requirement should be 10 mg. Pantothenic acid has no potential dangers and no significant physiological reactions are known from the state of the art. (13)

There is no hint for the assumption that pharma-pantothenic acid is taken orally. Older reports state no reactions after a daily portion of 5-20 g. Upon that the view is based, that pantothenic acid has no reaction potential. (13) Nowadays, young people are orally intaking 5-10 g pantothenic acid capsules for weeks for the treatment of acne.

After such intake CoA→Acetyl-CoA→Acetylcholinbuilding-followreactions should be expected in smooth muscles. A small group of the large amount of people who treat disease by themselves report that there occurred side effects as loose stool or stomach irritations, flatulence or hunger a big appetite. These side effects could be short acetylcholine-building excess reactions. The pre takeover existence of pantothenic acid-CoA is assumeable only with the effects of the reaction result steps of CoA. It is very likely that the pharma-substance pantothenic acid, pH 7-8, is deactivated in the stomach milieu under pH 1-2. Generally, the non-protected oral intake of pantothenic acid-CoA probably does not take place in stomach.

The invention is based on the following surprising finding: After treatment of chronic fatigue, muscle weakness/muscle pain, weakness and respiratory weakness with 140 mg of the Acetylcholinersteraseinhibitor Neostigminbromid, late at night, at breakfast, no response until then, 10 min after the first bite adverse reactions, weeping nose, watery eyes, urination, body trimming started, which subsided after 15 min. Immediately afterwards, response experiment to sublingual/buccal applied 2 g-Panthenol Tbl. was executed. After 10 min the same adverse reaction started with a similar duration. After its decay and continuation of the breakfast with bread the same reactions in bladder, womb and bowel 10 minutes later repeated with time shortened duration. Then after anew sublingual/buccal application of 2 g-Panthenol Tbl. the same adverse reaction started, but delayed and lasted for less. The 140 mg of the Acetylcholinersteraseinhibitor Neostigminbromid were still effective after 6 indifferent hours.

### The Hypothetical Surplus Building of Acetylcholine After Intake of Pantothenic Acid-Coenzym A in Experiment

Acetylcholine, the natural excitation transmitter from the nerve to the muscle effects striped muscles in concentrations of 10⁻⁶ g/l, smooth muscles already in concentrations of 10⁻¹⁶ g/l. (4) These “adverse” reactions are contractions of smooth muscles of lachrymal gland, bladder, stomach, intestine, caused by there suddenly synthesized acetylcholine, which exceeds the Response threshold that is 10⁻¹⁶ grams per liter of acetylcholine in smooth muscles:
a) within 10 minutes of ingestion into the stomach and after the end of reaction;
b) within 10 minutes of sublingual/buccal absorption of 2000 mg Tbl Panthenol, while Acetylcholinersterase-inhibition amongst 140 mg Neostgminbromid is existing. This suddenly and surprisingly occurred adverse cholinergic reactions are beyond the acetylcholine increase also evidence of ACH precursers CoA→Acetyl-CoA. The comparable reactions show that the stomach Pantothenic-CoA from food absorbs, nutrient-dependent about 10³-10⁴ times the amount of the previous assumptions. (13)

The core point of the invention is that the uptake of a pantothenic acid-CoA equivalent from food is not 10 mg per day, but more than 10³ times of this amount to provide the human organism optimally. It was also a surprising finding that the cholinergic Panthenol-CoA uptake reaction from food was taking place within a few minutes. It is therefore suggested that pantothenic acid-CoA immediately arrives into the mitochondria and becomes effective in the citric acid cyclus through the mucous membranes and/or uptaked in the stomach. The enormous disparity between minimal availability of pantothenic acid-CoA existence and the multi-varied requirement of acetyl-CoA in the CoA pathway of the citic acid cyclus was bypassed by the construction of a recombination of CoA.

In reality the role of coenzyme A has to be changed from the regenerating catalyst to a consumption factor, which reacts reaction limiting in reduced availability as a key factor in the mitochondria. The metabolic activity capacity of the organism is depending on the mass of the available Pantothenic-CoA deposits in the pool of mitochondria.

Muscle weakness and pain, weakness, respiratory weakness, mental exhaustion, and gastric problems have mainly been treated with pantothenic acid-CoA,-buk, vitamins and amino acids.

After improvement and discontinuation of the treatment the symptoms returned after a short interval. Ultimately, the problems could be reduced and neutralised through re-treatment. Only with the normalization of the function of the digestive tract, the absorption of pantothenic acid-CoA from food in the stomach could be reproduced, and the treatment was dispensable.

New is the uptake
of pantothenic acid from the stomach,
of pantothenic acid and the synthesis of Acetylcholin within 10-15 minutes,
of pantothenic acid from food about 10-100 g daily,
Pantothenic acid in an unknown way.

Also new is that diseases of the stomach pantothenic acid uptake hinder or diminish, as well as the presence of a potential deposit of a few kilograms of pantothenic acid in the form of CoA in the mitochondria, the tissue in the muscles, what from the metabolic activity is supplied and therefore the metabolic activity is depended on the availability of pantothenic acid->CoA. Thereby pantothenic acid->CoA is a consumption factor, whose decreased availability limits and exhausts actions and reactions as a key factor. This is reflected in the varying manifestations of mental and physical life and health state, in emotion, sexuality, creativity, memory, activity, movement, work, endurance, fatigue, relaxation, exhaustion, illness, convalescence, infirmity; variants of metabolic activity.

In daily life, power tires and condition exhausts from meal to meal. The teaching is that food and pantothenic acid is absorbed in the stomach in the small intestine after digestion. Time interval therefore is approximately 3 hours. However, 15 minutes eating break is enough to be powerful for 3-4 hours again. In addition and prior to the conventional digestion apparently this uptake of CoA agent takes place, from that this branch of the metabolic activity prior is supplied, which controls the nerve activity of emotion, sexuality, creativity, memory, activity, movement, work, and perseverance. This branch of the nerve activity control “consumes” the largest amount of the acetyl-CoA-CoA agent in mitochondria. This is firstly experienced with restrictions when deficiency occurs. Within 15 minutes, the stomach transfers pantothenic acid-CoA->Acetyl-CoA from food into the mitochondrial pool, applicable with it's full performance potential. Occasionally after rich meals acetylcholine surplus reactions occurs and show themselves as the briefly weeping eyes and watery nose.

To maintain or restore a stable metabolic rate a compound containing pantothenic acid-CoA is provided. Preferably, this compound contains large amounts of pantothenic acid, pantothenic acid-CoA or its precursors, particularly preferred in a daily dose of 10-100 g. The invention also concerns to a process for maintaining and improving metabolic activity, characterized by the uptake of large amounts of pantothenic acid, pantothenic CoA or its precursors.

The compensation of CoA deficiency in weakness in metabolic activity through addition of pantothenic acid-CoA enhances CoA->Acetyl-CoA balance in the tissues, muscles, in mitochondria, what from metabolic activity is supplied and/or supplements a lack of the uptake of pantothenic acid-CoA from the food through the stomach.

Disturbed gastric functions and/or depletion of mitochondrial reserves in Myasthenia, autism, chronic fatigue syndrome appears in the fading of the yellowing of the urine. Successful supply of pantothenic acid-CoA intensifies the yellowing. The current optimum, the proof of the success of the pantothenic acid-CoA treatment is reached when harmless acetylcholine surplus reactions responses in the bladder, stomach or intestines occur.

In diseases of the stomach and the digestive tract generally the uptake of all food agents is affected. Besides Pantothenic-CoA also the supporting compensation of the deficiency of amino acids, vitamins, etc by the coupled application of those substances is proposed. An additional application of Gingko preparations is also proposed.

Application forms for the administration of pantothenic acid, CoA Pantothen or precursors thereof, in combination with vitamins, amino acids, fatty acids, phospholipids, coenzymes, trace elements, bioactive substances, minerals and/or gingko, if applicable, are:

a) For the immediately need in weakness—fatigue:

Contact with the oral mucosa, preparation as quid, chewing lozenge, lozenge, and/or deposit;

b) through the contact with the small intestine after gastric passage protected as Gastro preparation in size from small-to capsule, pill, for example, by being incorporated into a gel, in pudding, in an aqueous carrier suitable and comparable forms with variable solution properties for a small point concentration;

c) dermal contact from pantothenic acid carrier foils, implants, etc.;

d) anal, for example as a suppository, etc.

Autistic children often suffer from gastric problems and chronic diarrhea. Due to the injury of the digestive tract, the pantothenic acid-CoA input property from food of the stomach is disrupted, with the result of chronic weakness in metabolic activity, autism in this case. The reduction in the availability of pantothenic acid-CoA, acetyl-CoA, acetylcholine chain hits the children in their most damageable and most vulnerable period of their mental development. The cause of injury to the stomach, which leads to this disease, is unknown. The risks posed by pesticides and preservative in food are obvious. Chemical exposure or other media near to the ground which the children are in direct contact, such as parquet, carpets, laundry, entry from the outside by dogs, etc. are proposed to this default.

These could be sources of contamination with wood preservatives, insecticides, mould, impregnation of flooring, spray pesticides against current infestation, infectious materials from domestic and wild animals. The harmful dose of uptake with breath just from the amount in the mouth of infants who are many years in positions close to these sources is summarized from the smallest amounts, and is exponentially higher than in 1.80 m height. Adverse reactions to vaccines or its preservatives, for example mercury, are suspected to cause autism. Autism, chronic fatigue syndrome (CFS) and the variety of gastric diseases and symptoms are still not curable until today. (5)

Dr.John Chia did find a with enteroviruses infected stomach in his son Andrew, who was affected by chronic fatigue syndrome CFS as a child. (6) After he succeeded in eliminating the enterovirus infection (i.e. by restoring the pantothenic acid-CoA uptake capacity of the stomach) Andrew recovered completely (7)

For the treatment of autism/CFS, chronic metabolic performance weakness and gastric symptoms, the Pantothenic-CoA complex according to the invention is provided. It repairs the deficit in the uptake of Pantothenic-CoA caused by gastric problems and prepares the restoration of normal gastric functions.

Patients harmed by Alzheimer's disease, AD, experienced an improvement of their condition after administration of acetylcholine inhibitors Tacrine, Donepezil, Galantamine Rivastigmin or Galantamin. (8) The side effects of nausea, vomiting, diarrhea and cramps are contractions of smooth muscles of stomach, intestine, bladder, that especially may occur after a meal . Then the acquisition of pantothenic acid-CoA from the dish begins immediately in the stomach. When the CoA-acetyl-CoA-acetylcholine increase reaches the response threshold of 10⁻¹⁶g/l in smooth muscles, they contract. In the report references to meals before the start of the reactions as well as their duration are lacking. Pantothenic acid-CoA treatment of AD provides a real variable compensation of ACH deficiency, avoids the necessarily occuring ACH exhaustion by the use of ACHE inhibitors and improves the state of existing AD without causing adverse reactions.

Soon after the return of the soldiers of Operation Desert Storm in Kuwait and Iraq 1991, a part of the veterans were infested by a number of chronic diseases, known as Gulf War Syndrome or Gulf War illnesses (GWI) against which no treatment has been found until today. (9) In some cases, the illness spread over to the family, wife and children. Chronic fatigue syndrome CFS, mental and gastric symptoms show weakness in metabolic activity in permanent manner. This weakness is a consequence of an affection of the digestive tract whereby the Pantothenic-CoA intake capacity from food of the stomach is disrupted. Regardless of the type of infection or poisoning the damage of the digestive tract resulted from, the supply of Pantothenic-CoA complex according to the invention improves the CoA-acetyl-CoA balance in the tissue, in the mitochondria, what from the metabolic performance is fed, repairs the Pantothenic-acid-CoA intake deficit caused by gastric problems and prepares the restoration of normal gastric functions.

The dietary supplement according to the invention is therefore also provided for the treatment of the so-called Gulfwar illness and related disorders.

Various antibiotics, aspirin and other drugs cause “stomach problems”, to the point of myasthenic phenomena, i.e. they affect the digestive tract. A mixture or a combination of pantothenic acid-CoA with these substances ensures a better long-term compatibility.

For a further verification the following observations are shown:

In the groups one to three and five constant natural acetylcholine surplus reactions occur after food intake, the experiment four was observed in 1991. Sudden acetylcholine surplus reactions after Pantothenic-CoA intake from food, adverse, cholinergic reactions after food intake befall:

1. Baby's, when they “spit” after feeding, when their stomach suddenly empties after feeding. Some additionally suffer from sudden diarrhea.

2. Children, often girls, not rarely vomit after eating.

3. Primates, like monkeys, vomit after eating lots of food.

4. 1991, the Iraq war. Thousands of young healthy men and women, soldiers, reported that their physis showed adverse cholinergic reactions after ingestion of pyridostigmine bromide (PB), ACH-Esterase inhibitor: (“side effects” of PB including nausea, vomiting, abdominal Cramps, diarrhea, increased salivation, sweating, muscle Cramps), they triggered forced Acetylcholin surplus reactions.

5. Dog, the pet, frequently vomits after feeding

Various antibiotics, aspirin and other drugs produce “stomach problems”, myasthene appearances. A mixture or other combination of pantothenic acid analogues with these, many substances provides for a significant better tolerability.

According to invention a dietary supplement comprising 85-99% Pantothenic acid, it's predecessors, its derivatives and/or its salts and other supplements if applicable, is provided. Preferably calcium pantothenate is used as a salt. In another preferred embodiment of the invention Pantethine is used as Pantothenic acid. It is produced by the company Daiichi Pharmaceutical Co., Ltd, as Pantesin.

Furthermore, subject of the invention is a compound for producing a preparation for the treatment of weakness in metabolic activity comprising 85-99% Pantothenic acid and/or its salts and other supplements if applicable, whereby calcium pantothenate and/or Pantethine is preferably used as a salt.

In a preferred version, the agent according to invention is enteric-coated provided.

As further additives vitamins, amino acid, fatty acids, phospholipids, coenzyme, micronutrients, bioactive substances, mineral nutrients, and/or gingko are used. The dietary supplement is used for the treatment of weakness in metabolic activity. Furthermore, it is also suitable for the treatment of chronic fatigue, weakness, respiratory weakness, for the improvement of mental and physical life and health state, in particular emotion, creativity, memory, regeneration, activity, sexuality, exercise, work, endurance and/or suitable convalescence.

Especially in the appearance of changing mental and physical life and health state, in emotion, sexuality, creativity, memory, activity, movement, work, endurance, fatigue, relaxation, exhaustion, illness, convalescence, infirmity, variants of life are represented in their metabolic capabilities, their weakness in metabolic activity. The mental-sexual complex, most sensitive in deficiency of all, first suffers from starting weakness in metabolic activity and reduction of ability and endurance.

So the dietary supplement for treatment of periodic womanly weakness and for treatment of a deficit in pantothenic acid is particularly age dependent suitable.

The mitochondria's dysfunction as a result of long continuing under-supply of CoA, caused by bacterial, viral, chemical damages of the digestive tract as a result of disturbed, diminished O2-uptake and O2-transfer and/or congestion, leads to the disintegration of metabolism.

The balance of the lack of CoA by supply of Pantothenic acid-CoA in the suggested composition causes more than only the balance of a topical lack.

Balancing supply remedy deficiencies of process substances of metabolism and cures fatal dysfunctions, defective reactions, disorders, defective degradations, dysplasias, ultimately illnesses of the organism linked to:
cancer,
low immunity,
autoimmune reaction, inflammation,
rheumatic disease,
cardiac output weakness,
allergy, pyrexia,
joint attrition, arthrosis, arthritis,
asthma,
masculine and feminine fertility disorder, sperm immobility,
depression
burnout syndrome,
degradation of capacity and concentration, exhaustion,
respiratory difficulties, worsen O2-utilisation under contaminated breathable air,
impact of ionising rays
impact of electromagnetic fields on stomach functions,
hair disease.

In a further form of execution the supplement according to the invention is suitable for prevention and/or treatment of cancer, low immunity, autoimmune reaction, inflammation and/or rheumatic diseases. It is further applicable in case of cardiac output weakness, allergies, arthritis, joint attrition, arthrosis, asthma, masculine and feminine fertility disorder, sperm immobility. In a further form of execution the supplement according to the invention is suitable for the prevention and treatment of depression, burnout syndrome, degradation of capacity and/or concentration, exhaustion, respiratory difficulties, worsen O2-utilisation under contaminated breathable air, impact of ionising rays, impact of electromagnetic fields on stomach functions and/or hair disease.

It is further applicable for the treatment of erectile dysfunction, miss sensations in the sexual area, deficient related perception functions, delusions, childhood problems, developmental disorders, behavioural disorders, inflammation, wear-articular disorders, contraction weakness, restless-legs-syndrome (RLS), incontinence, psychological problems and/or migraine. Furthermore it improves the tolerability of stomach affecting drugs.

In a specific embodiment it is applicable for the treatment and prevention of damages caused by the ingestion of Pyridostigminbromid and/or the treatment of Gulf War disease.

In a further form of execution the supplement according to the invention is suitable for the treatment of Tinnitus suitable. Tinnitus also results from weakness in metabolic activity and belongs to the first signs of further and serious secondary diseases.

It was also found that the effectiveness of insulin doses in the treatment of diabetes mellitus could significantly be improved by combined use with the compound according to the invention. The effect of insulin is enhanced and effective for a longer term. Therefore the compound is also to use for treatment of diabetes in combination with an insulin therapy.

In a further form of execution is useable to improve the condition of Alzheimer's disease (AD) patients.

Also, it is applicable for supporting the treatment of infections, infectious diseases, fever, HIV, carcinomas and/or treatment in psychiatry and to prevent and to support the treatment of autism and/or autistic disorders.

It improves the gastric function and leads to an improvement of the effect of antibiotics, when they are applied orally.

The compensation of CoA-Acetyl-CoA-deficiency in mitochondria by addition of the compound needs time to “recharge” the Agent stock to achieve regeneration and improvement. This process is comparable to the convalescence after a serious illness, when the status of power slowly improves by the own intake of CoA. In disturbed CoA intake under gastric problems, the “recharge” by supplement is more longsome.

With profound weakness in metabolic activity an immediate improvement is not expected. After initial improvements weeks can pass until constant success takes place. The compound is to apply in variable quantities, appropriate to the condition, in individual doses of 1 g to 10 g over several times a day prior to or after the meals.

The occurrence of side effects, adverse reactions in smooth muscles, marking the optimum of the intake, the compound is easy to reduce.

Application possibilities for the compound are oral, buccal, sublingual, anal, intramuscular, intravenous or intra-arterial administration, other possibilities are also conceivable.

Preferably, the compound is applied as a quid, a chewable tablet, jelly, pudding, lozenge, or deposit.

The intake of the compound takes place over contact with the oral mucosa.

The compound is preferably fixed in a predominantly edible carrier, even with taste, in any suitable form. The carrier may consist of jelly, fruit gums, starch, vegetable matter or an inert material, i.e. tissue pocket. The preparation is portioned and packaged in a durable form.

Inclusion of the compound by oral intake:

In a known way, the compound is portioned in any form and displaced into a gastro resistant condition. In the about size of rice, lentils, peas beans, the compound can be used loosely with any liquid carrier.

Improved handling and dosage can be achieved with packaged preparations. These contain the gastro resistant compound in a liquid carrier, such as pudding, soup, milk food, ready for application or separately for mixture before using in different concentrations. In a preferred embodiment, the provision/use of the product takes place in assembled battery packs appropriate for weeks/month duration of the treatment.

Another special embodiment is determined by the incorporation of pantothenic acid, their predecessors, their derivatives and/or their salts to beverages. Therefore suitable are soft drinks, especially the so-called functional drinks, wellness drinks or power drinks, which are of increasing popularity in present time. Therefore the constitutional benefits of the compound according to the invention can be used simply everyday. In addition, an enhanced energy drink is provided, which naturally increases the metabolic activity and acts constitutionally.

The active content of the drink consists preferably of primarily small-coated gastro resistant additions of pantothenic acid, its precursors, their derivatives and/or their salts.

Also object of the invention is a method for producing the dietary supplement as well as the compound.

Following, the invention is explained in examples.

### EXAMPLE 1

Babies like to “spit” after feeding (a cholinergic reaction). From the milk food, the stomach immediately adopts Pantothenic-CoA into the metabolism. If the formation of acetylcholine in the (smooth) muscle stomach exceeds the reacting value of 10⁻¹⁶ g/l in the muscles, it contracts. Some babies have such a strong intake capacity of CoA that they suffer from diarrhea, additionally to the sudden contraction of the stomach. Sometimes even small children vomit after meals, mostly girls.

### EXAMPLE 2

In Iraq War in 1991 250 000-300 000 U.S. and coalition troops ingested the active ingredient Pyridostigminbromid (PB) as protection against Iraqi poison gas attacks. Young healthy men and women, soldiers, reported about the “self-experiment” in which after their ingestion of 30 mg PB, ACH Esterase Inhibitor their constitution showed adverse reactions, they triggered forced acetylcholine surplus, which were evident in the contraction of smooth muscles, in the strongest form as nausea, vomiting, stomach cramps, diarrhea, urination, increased salivation, sweating and muscle spasm. These effects occurred shortly after ingesting the tablets, and disappeared with discontinuation of medication.

Many soldiers had taken the PB tablets together with food. The pantothenic acid CoA adoption of the stomach from the food was high, as the PB inhibited the acetylcholinesterase. In stomachs with acetylcholine formation from food massive acetylcholine surplus reactions occur, together with the described effects of contraction of the stomach, bladder, glands. The connection between the ACH-accumulation-surplus and the food intake is not known. (9) (10).

### EXAMPLE 3

Primates, like monkeys, vomit after plenty of food eaten. The pet dog vomits often after feeding. The dependence of the stomach contractions on previously recorded feeding is evident.

All characteristics described in the preceding description and the following claims, both individually and in any combination can be significant for realizing the invention in its various embodiments.

### REFERENCES

1. Nicolson, G. L. Ph.D, Lipid Replacement as an Adjunct to Therapy for Chronic Fatigue, Anti- Aging and Restoration of Mitochondrial Function, The Journal of the American Nutraceutical Association Vol. 4, No. 1, 2001
2. Nicolson, G. L. Ph.D, Chronic Fatigue, Aging, Mitochondrial Function and Nutritional Supplements, Townsend Letter for Doctors and Patients, July, 03
3. Keil, Uta, Schlüsselfunktion der Mitochondrien in der Pathogenese der Alzheimer Demenz. Dissertation, Frankfurt am Main 2005
4. dtv Atlas zur Biologie 2. edition 1969, Vol. 2, S.375, Der tetanische Muskel unter Curare; Acetylcholin, der natürliche Erregungsüberträger vom Nerv auf den Muskel, wirkt auf gestreifte Muskeln in Konzentrationen um 10⁻⁶ g/l, auf glatte schon bei 10⁻¹⁶ g/l.

5. J. Madeleine Nash, The Secrets of Autism, The number of children diagnosed with autism and Asperger's in the U.S. is exploding. Why?mailto:daily@timeinc.net, TIME MAGAZINE, ISSUE MAY 6, 2002
6. Chia ,John kai-sheng and Chia, Andrew Y. Chronic fatigue syndrome is associated with chronic enterovirus infection of the stomach, J. Clin. Pathol. Published Online First: 13 Sep. 2007.
7. Sep. 13. 2007 HealthDayNews woman's Health.gov. The Federal Government Source for Woman's Health Information.
8. Cummings ,J. L. Treatment of Alzheimer's disease. Dementia, Vol 3, No 4,
9. Research Advisory Committee on Gulf War Veterans' Illnesses, Gulf War Illness and the Health of Gulf War Veterans. Additional Clinical and Research Findings, 263, Assessment of gastrointestinal symptoms and conditions. U.S. Department of Veteran Affairs Washington D.C., 2008
10. Keeler, J. R. ,Hurst C. G and. Dunn M. A, Pyridostigmine used as a nerve agent pretreatment under wartime conditions, Jama Vol. 266 No.5, August 7
11. Myhill, S. Booth N, E. McLaren-Howard, J. Chronic fatigue syndrome and mitochondrial dysfunction. Int J Clin Exp Med (2009) 2, 1-16
12. Bains, W. Treating Chronic Fatigue states as a disease of the regulation of energy metabolism. edical hypothesis 2008 Delta G Ltd, 37 The Moor, Melbourn, Royston, Herts SG8 6ED, UK
13. Food commission of the EU SFC 1992, 2002. Food and Nutrition Board (USA), IMO 2000, EVM (UK) 2003

1. A nutritional supplement comprising 85-99% of at least one member of the group consisting of pantothenic acid, its predecessors, its derivatives and its salts.

2. A preparation comprising a member of the group consisting of 85-99% pantothenic acid and its salts.

3. The preparation of claim 2, wherein said preparation is suitable for the treatment of weakness in metabolic activity and associated symptoms.

4. The preparation of claim 2, wherein said preparation is suitable for the treatment of at least one member of the group consisting of chronic fatigue and weakness, respiratory weakness, age-related deficiency in pantothenic acid, for the treatment and prevention of damages caused by the ingestion of Pyridostigminbromid, and improvement of mental and physical life and health sensitivities.

5. The preparation of claim 2, wherein said preparation is suitable for the treatment of at least one member of the group consisting of periodic womanly weakness, migraine, Gulf War illness, Tinitus, diabetes in combination with an insulin therapy, symptoms of poisoning by pesticides, symptoms of poisoning by insecticides, from injury and poisoning, especially in agriculture, for the improvement of the tolerability of gastro disturbing drugs, improvement of the condition of Alzheimer's disease (AD) patients, for the reduction of ACHE inhibitors caused side effects in Alzheimer's disease (AD) patients, for the treatment of erectile dysfunction, missensations in the sexual area, deficient related perception functions, delusions, infantile problems, developmental disorders, behavioural disorders, inflammation, rheumatic disorders, wear-articular disorders, contraction weakness, restless-legs-syndrome (RLS), incontinence, psychological problems and migraine.

6. The preparation of claim 2, wherein said preparation is suitable for the treatment of at least one member of the group consisting of infections, infectious diseases, fever, HIV, Carcinome, treatment in psychiatry, for the prevention and the support of the treatment of autism and/or autistic disorders and/or for the improvement of gastric function and the improvement of the effect of antibiotics applied orally.

7. The treatment of claim 2, wherein said treatment is suitable for at least one member of the group consisting of prevention and accompanying treatment of at least one member of the group consisting of cancer, low immunity, autoimmune reaction, cardiac output weakness, allergies, arthritis, joint attrition, arthrosis, asthma, for improvement of masculine and feminine fertility, improvement of sperm mobility, depression, burnout syndrome, degradation of activity capacity and/or concentration, impairment of endurance and memory, impairment O2-utilisation under contaminated breathable air, impact of ionising rays, impact of electromagnetic fields on stomach functions and hair disease.

8. The treatment of claim 2, wherein the treatment is applied by a route selected from the group consisting of oral, buccal, sublingual, anal, intramuscular, intravenous and intra-arterial.

9. The treatment of claim 2, further comprising a carrier.

10. Procedure for the manufacture of a nutritional supplement according to claim 1.

11. Procedure for the manufacture of a compound according to claim 2.

12. A method for improving metabolic activity, comprising identifying a patient in need of improvement of metabolic activity and administering an effective amount of the nutritional supplement of claim 1 to that patient.

13. A method for improving metabolic activity comprising identifying a patient in need of improvement of metabolic activity and administering an effective amount of the treatment of claim 1 to that patient.

14. The nutritional supplement of claim 1, wherein said nutritional supplement is suitable to improve the metabolic activity of a patient.

15. Procedure for the treatment of weakness in metabolic activity, comprising ingesting large daily doses of pantothenic acid, its precursors, their derivatives and/or their salts.
